(19) United States
(12) Patent Application Publication
(10) Pub. No.: US 2009/0118559 A1
(43) Pub. Date: May 7, 2009

(54) Oxidic Metal Composition, Its Preparation And Use As Catalyst Composition

(75) Inventors: Dennis Stamires, Dana Point, CA (US); Paul O'Connor, Hoevelaken (NL); William Jones, Cambridgeshire (GB)

Correspondence Address: Albemarle Netherlands B.V.;Patent and Trademark Department, 451 Florida Street, Baton Rouge, LA, 70801, US

(73) Assignee: ALBEMARLE NETHERLANDS B.V., Amersfoort (NL)

(21) Appl. No.: 11/915,698

(22) PCT Filed: Jun. 2, 2006
(86) PCT No.: PCT/EP06/62897

§ 371 Date: Sep. 15, 2008

Related U.S. Application Data
(60) Provisional application No. 60/687,309, filed on Jun. 6, 2005.

(51) Int. Cl. B01J 23/10; B01J 29/06; B01J 23/745; B01J 23/06; C07C 7/20

(52) U.S. Cl. 585/850; 502/65; 502/304; 502/336; 502/342; 585/852

(57) ABSTRACT

Oxidic composition consisting essentially of oxidic forms of a first metal, a second metal, and optionally a third metal, the first metal being either Fe or Zn and being present in the composition in an amount of from about 5 to about 80 wt %, the second metal being Al and being present in the composition in an amount of from about 5 to about 80 wt %, the third metal being selected from the group consisting of Mo, W, Ce, and V, and being present in an amount of from 0 to about 17 wt %—all weight percentages calculated as oxides and based on the weight of the oxidic composition, the oxidic composition being obtainable by (a) preparing a physical mixture comprising solid compounds of the first, the second, and the optional third metal, (b) optionally aging the physical mixture, without anionic clay being formed, and (c) calcining the mixture. This composition is suitable for use in FCC processes for the reduction of SOₓ emissions from the regenerator and for the production of sulphur-lean fuels and has only a minimised influence on the zeolite's hydrothermal stability.

The present invention relates to an oxidic composition consisting essentially of oxidic forms of a first metal, a second metal, and optionally a third metal and its use in catalytic processes, such as fluid catalytic cracking (FCC).

EP-A 0 554 968 (W.R. Grace and Co.) relates to a composition comprising a coprecipitated ternary oxide comprising 30-50 wt % MgO, 30-50 wt % Al₂O₃, and 5-30 wt % La₂O₃. The composition is used in a fluid catalytic cracking process for the passivation of metals (V, Ni) and the control of SOₓ emissions from the regenerator of the FCC unit.

U.S. Pat. No. 6,028,023 discloses the preparation of hydrotalcite-like compounds from MgO and Al₂O₃. These compounds are prepared by (a) preparing a reaction mixture comprising an Mg-containing compound and an Al-containing compound, thereby forming either a hydrotalcite-like compound or a non-hydrotalcite-like compound, followed by calcination and rehydration. The resulting compound is used in an FCC process for the reduction of SOₓ emissions.

The disadvantage of the above compositions is that when they are incorporated into a zeolite-containing FCC catalyst, they have a negative effect on the zeolite's hydrothermal stability.

The object of the present invention is to provide a composition which is suitable for use in FCC processes for the reduction of SOₓ emissions from the regenerator and for the production of sulphur-lean fuels, while at the same time this composition has a minimised influence on the zeolite's hydrothermal stability.

The present invention relates to an oxidic composition consisting essentially of oxidic forms of a first metal, a second metal, and optionally a third metal, the first metal being either Fe or Zn and being present in the composition in an amount of 5-80 wt %, the second metal being Al and being present in the composition in an amount of 5-80 wt %, the third metal being selected from the group consisting of Mo, W, Ce, and V, and being present in an amount of 0-17 wt %—all weight percentages calculated as oxides and based on the weight of the oxidic composition, the oxidic composition being obtainable by
a) preparing a physical mixture comprising solid compounds of the first, the second, and the optional third metal,
b) optionally aging the physical mixture, without anionic clay being formed, and
c) calcining the mixture.

That the oxidic composition “consists essentially of” oxidic forms of a first metal, a second metal, and optionally a third metal means that the oxidic composition does not contain any other materials in more than insignificant trace amounts.

Step a)

The oxidic composition according to the present invention is obtainable by a process which involves as a first step the preparation of a physical mixture of solid compounds of the first metal (Zn or Fe), the second metal (Al), and the optional third metal (Mo, W, Ce, or V). This physical mixture is prepared by mixing the solid compounds, either as dry powders or in a liquid, to form a suspension, a sol, or a gel.

The physical mixture must contain solid metal compounds. This means that when preparing the physical mixture in a liquid, the metal compounds do not dissolve in this liquid, at least not to a significant extent. In other words, if water is used to prepare the physical mixture, water-soluble metal salts should not be used as the metal compounds.

On the other hand, if the physical mixture is prepared by dry mixing the metal compounds, then water-soluble salts can be used.

The preferred compounds of the first, second, and third metals are oxides, hydroxides, carbonates, and hydroxycarbonates, because these compounds are generally water-insoluble and do not contain anions that decompose to harmful gases during calcination step c). Examples of such anions are nitrate, sulphate, and chloride, which decompose to NOₓ, SOₓ, and halogen-containing compounds during calcination.

Suitable zinc compounds include zinc oxide, zinc basic carbonate, zinc acetate, zinc acetate hydrate, zinc citrate hydrate, zinc oxide hydrate, and zinc stearate. Suitable iron compounds include iron ores such as goethite (FeOOH), bernalite, feroxyhyte, ferrihydrite, lepidocrocite, limonite, maghemite, magnetite, hematite, and wustite, and synthetic iron products such as synthetic iron oxides and hydroxides, iron carbonate, iron bicarbonate, and iron hydroxycarbonate.

Suitable aluminium compounds include aluminium alkoxide, aluminium oxides and hydroxides such as transition alumina, aluminium trihydrate (gibbsite, bayerite) and its thermally treated forms (including flash-calcined alumina), alumina sols, amorphous alumina, (pseudo)boehmite, aluminium carbonate, aluminium bicarbonate, and aluminium hydroxycarbonate. With the preparation method according to the invention it is also possible to use coarser grades of aluminium trihydrate such as BOC (Bauxite Ore Concentrate) or bauxite.

Suitable molybdenum compounds are molybdic acid, potassium molybdate, sodium molybdate, ammonium molybdate, and molybdenum acetate.

Suitable tungsten compounds are sodium tungstate, ammonium metatungstate, and tungstic acid.

Suitable cerium compounds are cerium acetate, cerium oxalate, cerium citrate, and cerium phosphate.

Suitable vanadium compounds are vanadium oxalate and ammonium metavandate.

The weight percentage of the first metal in the precursor mixture and in the resulting oxidic composition is 5-80 wt %, preferably 10-50 wt %, calculated as oxide and based on dry solids weight.

The weight percentage of the second metal in the precursor mixture and in the resulting oxidic composition is 5-80 wt %, preferably 20-60 wt %, calculated as oxide and based on dry solids weight.

The weight percentage of the third metal in the precursor mixture and in the resulting oxidic composition is 0-17 wt %, preferably 3-15 wt %, calculated as oxide and based on dry solids weight.

The physical mixture may be milled before calcination, as dry powder or in suspension. Alternatively, or in addition to milling of the physical mixture, the compounds of the first, second, and/or third metal can be milled individually before forming the physical mixture. Equipment that can be used for milling includes ball mills, high-shear mixers, colloid mixers, kneaders, electrical transducers that can introduce ultrasound waves into a suspension, and combinations thereof.

If the physical mixture is prepared in aqueous suspension, dispersing agents can be added to the suspension, provided that these dispersing agents are combusted during the calcination step. Suitable dispersing agents include surfactants, sugars, starches, polymers, gelling agents, etc. Acids or bases may also be added to the suspension.

Step b)

The physical mixture can be aged, provided that no anionic clay is formed.

Anionic clays—also called hydrotalcite-like materials or layered double hydroxides—are materials having a crystal structure consisting of positively charged layers built up of specific combinations of divalent and trivalent metal hydroxides between which there are anions and water molecules, according to the formula

[Mm²⁺Mn³⁺(OH)_{2m+2n·}]X_{n/z}^{z-}·bH₂O

wherein M²⁺ is a divalent metal, M³⁺ is a trivalent metal, and X is an anion with valency z. m and n have a value such that m/n=1 to 10, preferably 1 to 6, more preferably 2 to 4, and most preferably close to 3, and b has a value in the range of from 0 to 10, generally a value of 2 to 6, and often a value of about 4.

Hydrotalcite is an example of a naturally occurring anionic clay wherein Mg is the divalent metal, Al is the trivalent metal, and carbonate is the predominant anion present. Meixnerite is an anionic clay wherein Mg is the divalent metal, Al is the trivalent metal, and hydroxyl is the predominant anion present.

If the formation of anionic clay is prevented, calcination (step c) results in the formation of compositions comprising individual, discrete oxide entities of the first, the second, and the optional third metal.

Formation of anionic clay during aging can be prevented by aging for a short time period, i.e. a time period which, given the specific aging conditions, does not result in anionic clay formation.

Aging conditions which influence the rate of anionic clay formation are the choice of the first and third metals, the temperature (the higher, the faster the reaction), the pH (the higher, the faster the reaction), the type and the particle size of the metal compounds (larger particles react slower than smaller ones), and the presence of additives that inhibit anionic clay formation (e.g. vanadium, sulphate).

Step c)

The precursor mixture, either aged or not, is calcined at a temperature in the range of 200-800° C., more preferably 300-700° C., and most preferably 350-600° C. Calcination is conducted for 0.25-25 hours, preferably 1-8 hours, and most preferably 2-6 hours. All commercial types of calciners can be used, such as fixed bed or rotating calciners. Calcination can be performed in various atmospheres, e.g, in air, oxygen, an inert atmosphere (e.g. N₂), steam, or mixtures thereof.

If necessary, the precursor mixture is dried before calcination. Drying can be performed by any method, such as spray-drying, flash-drying, flash-calcining, and air drying.

Use of the Oxidic Composition

The oxidic composition according to the invention can suitably be used in or as a catalyst or catalyst additive or sorbent in a hydrocarbon conversion, purification, or synthesis process, particularly in the oil refining industry and Fischer-Tropsch processes. Examples of processes where these compositions can suitably be used are catalytic cracking, hydrogenation, dehydrogenation, hydrocracking, hydroprocessing (hydrodenitrogenation, hydrodesulphurisation, hydro-demetallisation), polymerisation, steam reforming, base-catalysed reactions, gas-to-liquid conversions (e.g. Fischer-Tropsch), and the reduction of SOₓ and NOₓ emissions from the regenerator of an FCC unit. The oxidic composition according to the invention may also be used in biomass conversion processes.

In particular, it is very suitable for use in FCC processes for the reduction of SOₓ emissions and the production of fuels (like gasoline and diesel) with a low S and N content.

The oxidic composition according to the invention can be added to the FCC unit as such, or it can be incorporated into an FCC catalyst, resulting in a composition which besides the oxidic composition according to the invention comprises conventional FCC catalyst ingredients, such as matrix or filler materials (e.g. clay such as kaolin, titanium oxide, zirconia, alumina, silica, silica-alumina, bentonite, etc.), and molecular sieve material (e.g. zeolite Y, USY, REY, RE-USY, zeolite beta, ZSM-5, etc.). Therefore, the present invention also relates to a catalyst particle containing the oxidic composition according to the invention, a matrix or filler material, and a molecular sieve.

1. A composition consisting essentially of oxidic forms of a first metal, a second metal, and optionally a third metal, the first metal being either Fe or Zn and being present in the composition in an amount of from about 5 to about 80 wt %, the second metal being Al and being present in the composition in an amount of from about 5 to about 80 wt %, the third metal being selected from the group consisting of Mo, W, Ce, and V, and being present in an amount of from 0 to about 17 wt %—all weight percentages calculated as oxides and based on the weight of the oxidic composition, the oxidic composition being obtainable by
a) preparing a physical mixture comprising solid compounds of the first, the second, and the optional third metal,
b) optionally aging the physical mixture, without anionic clay being formed, and
c) calcining the mixture.

2. The composition according to claim 1 wherein the solid compounds of the first, the second, and the optional third metal are oxides, hydroxides, carbonates, or hydroxycarbonates.

3. The composition according to claim 1 wherein the first metal is present in an amount of from about 10 to about 50 wt %, calculated as oxide and based on the weight of the oxidic composition.

4. The composition according to claim 1 wherein the second metal is present in an amount of from about 20 to about 60 wt %, calculated as oxide and based on the weight of the oxidic composition.

5. The composition according to claim 1 wherein the third metal is present in an amount of from about 3 to about 15 wt %, calculated as oxide and based on the weight of the oxidic composition.

6. A catalyst particle comprising an oxidic composition consisting essentially of oxidic forms of a first metal, a second metal, and optionally a third metal, the first metal being either Fe or Zn and being present in the composition in an amount of from about 5 to about 80 wt %, the second metal being Al and being present in the composition in an amount of from about 5 to about 80 wt %, the third metal being selected from the group consisting of Mo, W, Ce, and V, and being present in an amount of from 0 to about 17 wt %—all weight percentages calculated as oxides and based on the weight of the oxidic composition, a matrix or filler material, and a molecular sieve.

7. (canceled)

8. A process for the conversion, purification or synthesis of a hydrocarbon comprising the step of contacting the hydrocarbon with an oxidic composition consisting essentially of oxidic forms of a first metal, a second metal, and optionally a third metal, the first metal being either Fe or Zn and being present in the composition in an amount of from about 5 to about 80 wt %, the second metal being Al and being present in the composition in an amount of from about 5 to about 80 wt %, the third metal being selected from the group consisting of Mo, W, Ce, and V, and being present in an amount of from 0 to about 17 wt %—all weight percentages calculated as oxides and based on the weight of the oxidic composition.

9. The process of claim 8 wherein the oxidic composition is used to reduce SOx emissions from an FCC regenerator.

10. The process of claim 8 wherein the oxidic composition is used to reduce S or N content of gasoline or diesel produced from an FCC process.
